(19) United States
(12) Patent Application Publication
(10) Pub. No.: US 2024/0010693 A1
(43) Pub. Date: Jan. 11, 2024

(54) PEPTIDES FROM HUMAN B-CASEIN THAT HAVE ANTI-BACTERIAL ACTIVITY

(71) Applicant: The Regents of the University of California, Oakland, CA (US)

(72) Inventors: Ishita M. SHAH, Roseville, CA (US); J. Bruce GERMAN, Davis, CA (US); David A. MILLS, Davis, CA (US); Carlito B. LEBRILLA, Davis, CA (US)

(21) Appl. No.: 18/035,267

(22) PCT Filed: Nov. 10, 2021
(86) PCT No.: PCT/US2021/058803

§ 371 Date: May 3, 2023

Related U.S. Application Data
(60) Provisional application No. 63/114,182, filed on Nov. 16, 2020.

(51) Int. Cl. C07K 14/47; A61K 47/12; A61P 31/04; A61K 47/02; A61K 47/22

(52) U.S. Cl. CPC C07K 14/4732; A61K 47/12; A61P 31/04; A61K 47/02; A61K 47/22

(57) ABSTRACT

An antibacterial polypeptide and methods of use are described.

### CROSS-REFERENCE TO RELATED PATENT APPLICATIONS

The present patent application is a U.S. National Phase Application Under 371 of PCT/US2021/058803 filed Nov. 10, 2021, which claims benefit of priority to U.S. Provisional Patent Application No. 63/114,182, filed Nov. 16, 2020, each of which is incorporated by reference for all purposes.

### SEQUENCE LISTING

The contents of the electronic sequence listing (081906-1282310-232710PC_SL.txt; Size: 48,471 bytes; and Date of Creation: Dec. 28, 2021) is herein incorporated by reference in its entirety.

### BACKGROUND OF THE INVENTION

Antibacterial peptides can provide an alternative to traditional antibiotics.

### BRIEF SUMMARY OF THE INVENTION

In some embodiments, an isolated polypeptide of 8-200 amino acids in length is provided that comprises an amino acid sequence that has 0, 1, 2 or 3 amino changes relative to RVMPVLKSPTIP (SEQ ID NO:1), RVMRVLKSPTIP (SEQ ID NO:3) or RVRPKLKSPRIP (SEQ ID NO:4) or a fragment thereof. In some embodiments, the amino acid sequence is RV(M/R)(P/R)(V/K)LKSP(T/R)IP (SEQ ID NO: 231). In some embodiments, the polypeptide is fused to a heterologous peptide sequence. In some embodiments, the amino acid sequence is SEQ ID NO:1, SEQ ID NO:3 or SEQ ID NO:4. In some embodiments, the polypeptide comprises TVYTKGRVMPVLKSPTIPFFDPQI (SEQ ID NO:2). In some embodiments, the amino acid sequence is KDTVYTKGRV (SEQ ID NO:5), TKGRVMPVLK (SEQ ID NO:6), TKGRVRPRLK (SEQ ID NO:7), VALARPKLPL (SEQ ID NO:8), VARRRPKLPL (SEQ ID NO:9), QRRPAIAINN (SEQ ID NO:10), KRRPAIAINN (SEQ ID NO:11), QRRPRIAINN (SEQ ID NO:12). In some embodiments, the polypeptide comprises a sequence selected from FIG. 19A, 19B, 19C or 19D (i.e., any one of SEQ ID NOS 16-230). In some embodiments, the polypeptide is 8-50, 8-150, 8-100, 8-30, 10-20, 12-20, 12-50, 12-100, or 12-150 amino acids amino acids in length.

In some embodiments, the polypeptide comprises one or more D-amino acids. In some embodiments, the polypeptide comprises a non-natural modification. In some embodiments, the modification is an amidation or glycosylation or PEGylation. In some embodiments, the modification occurs at the N- or the C-terminus of the polypeptide.

Also provided is a composition for administration to a human or animal, the composition comprising the polypeptide as described above or elsewhere herein. In some embodiments, the composition is an ointment or a gel or a mouthwash, or a rinse solution, or a nutritional beverage or an adherent coating. In some embodiments, the composition further comprises one or more of lactic acid, citric acid, malic acid, tartaric acid, phosphoric acid, acetic acid, propionic acid, acetic acid, butyric acid, indole lactic acid, indole acetic acid, indole propionic acid, indole acrylic acid, indole aldehyde, or indole ethanol.

Also provided is a method of administering the polypeptide as described above or elsewhere herein to a human or non-human animal in an amount sufficient to inhibit a bacterial pathogen in the human or non-human animal. In some embodiments, the administering comprises delivering the polypeptide to the human or non-human animal.

In some embodiments, the administering comprises delivering a polynucleotide encoding the polypeptide to the human or non-human animal, and the polypeptide is expressed in the human or non-human animal from the polynucleotide.

In some embodiments, the administering comprises delivering a cell comprising a heterologous polynucleotide encoding the polypeptide to the human or non-human animal, and the polypeptide is expressed in the cell, thereby delivering the polypeptide to the human or non-human animal.

In some embodiments, the administering is intravaginal, oral, rectal, intravenous, via inhalation, nasal, rectally, intraperitoneal, parenteral, intramuscular, subcutaneous, or transdermal.

Also provided is a polynucleotide (optionally isolated) comprising a promoter operably linked to a heterologous coding sequence, wherein the coding sequence encodes the polypeptide as described above or elsewhere herein.

Also provided is a method of making the polypeptide as described above or elsewhere herein, the method comprising expressing the polypeptide in a cell and harvesting the polypeptide from the cell.

### DEFINITIONS

An “expression cassette” refers to a nucleic acid construct that, when introduced into a host cell, results in transcription and/or translation of an RNA or polypeptide, respectively.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

The terms “peptidomimetic” and “mimetic” refer to a synthetic chemical compound that has substantially the same functional characteristics of a naturally or non-naturally occurring polypeptide, but different (though typically similar) structural characteristics. Peptide analogs are commonly used in the field as non-peptide active compounds (e.g., drugs) with properties analogous to those of a template peptide. Such non-peptide compounds are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987)). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., SEQ ID NO:1) such as found in a polypeptide of interest, but have one or more peptide linkages optionally replaced by a linkage selected from the group consisting of, e.g., —CH2NH—, —CH2S—, —CH2—CH2—, —CH═CH— (cis and trans), —COCH2—, —CH(OH)CH2—, and —CH2SO—. A mimetic can be either entirely composed of synthetic, non-natural analogues of amino acids, or, is a chimeric molecule of partly natural peptide amino acids and partly non-natural analogs of amino acids. A mimetic can also incorporate any amount of natural amino acid conservative substitutions as long as such substitutions also do not substantially alter the mimetic's structure and/or (e.g., anti-bacterial) activity.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an αcarbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as pyrrolysine, pyrroline-carboxy-lysine, and selenocysteine.

As used herein, the term “substantial identity” or “substantially identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Embodiments of the present disclosure provide for antimicrobial (e.g., antibacterial) peptides that are substantially identical to any of SEQ ID NO:1, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 OR SEQ ID NO:12.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

An algorithm that is suitable for determining percent sequence identity and sequence similarity is the BLAST 2.0 algorithm, described in Altschul et al. (1990) J. Mol. Biol. 215: 403-410 and based on that described in Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

The term “isolated,” when applied to a nucleic acid or protein, denotes that the nucleic acid or protein is purified to be essentially free of other cellular components with which it is associated in the natural state. It is often in a homogeneous or nearly homogeneous state. It can be in either a dry or aqueous solution. Purity and homogeneity may be determined using analytical chemistry techniques known and used typically in the art, e.g., polyacrylamide gel electrophoresis, high performance liquid chromatography, etc. A protein that is the predominant species present in a preparation is substantially purified. The term “purified” in some embodiments denotes that a protein gives rise to essentially one band in an electrophoretic gel. Typically, it means that a protein is at least 85% pure, e.g., at least 95% pure, or at least 99% pure.

A polynucleotide or polypeptide sequence is “heterologous” to a cell if it originates from a different cell, or, if from the same cell, is modified from its original form. For example, when a first amino acid sequence in a protein is said to be heterologous to a second amino acid sequence in the same protein, it means that the first amino acid is from a first cell or is non-naturally-occurring whereas the second amino acid is from a second cell or is modified from its original form.

As used in this specification and the appended claims, the singular forms “a,” “an,” and “the” include plural referents unless the context clearly dictates otherwise.

### BRIEF DESCRIPTION OF THE DRAWINGS

FIG. 1 depicts spot clearance assay results.

FIG. 2 depicts the human (β-casein amino acid sequence (SEQ ID NO:13).

FIG. 3 depicts the human αS1-casein amino acid sequence (SEQ ID NO:14).

FIG. 4 depicts the human κ-casein amino acid sequence (SEQ ID NO:15).

FIG. 5 depicts bacterial kill assay results.

FIG. 6 depicts HBCA2.C renders several clinically relevant pathogens non-viable in minutes.

FIG. 7 depicts additional bacterial strains sensitive to HBCA2.C at 20 mg/ml.

FIG. 8 depicts rapid killing of P. gingivalis by HBCA2 peptide.

FIG. 9 depicts direct kill assays of G. vaginalis and HBCA peptides.

FIG. 10 depicts that G. vaginalis is sensitive to HBCA2.C at 20 mg/ml.

FIG. 11 depicts direct kills assays of BV associated pathogens with HBCA2.C (mg/ml).

FIG. 12 depicts HBCA2.C has no effect on viability of select Lactobacilli.

FIG. 13 depicts HBCA2.C selectively targets G. vaginalis in co-cultures.

FIG. 14 depicts the presence of 10 mM D-lactate results in rapid killing of G. vaginalis by HBCA2.C at 20 mg/ml.

FIG. 15 depicts properties and activity of HBCA, HAS1CA and HKCA peptides.

FIG. 16 depicts activity of HBCA2 peptide against K. pneumoniae at 20 mg/ml and 10 mg/ml on TSA plates.

FIG. 17 depicts HBCA2.C spectra alterations at indicated pH.

FIG. 18A-B depict TEM images of K. pneumoniae (A) and L. rhamnosus (B) in the absence and presence of HBCA2.C.

FIG. 19A-C depict peptides as described herein including fragments of the antibacterial peptide HBCA2. FIG. 19D depicts peptides derived from HBCA105, HBCA110, HBCA110.1, HAS1CA12, HAS1CA12.1, HKCA64 and HKCA64.1

### DETAILED DESCRIPTION OF THE INVENTION

The inventors have discovered peptides (referred to herein as “HBCA1” and “HBCA2”) from human β-casein (hereinafter “casein”) that have antibacterial activity against a number of bacterial pathogens but is not substantially harmful to a number of bacterial flora that are not pathogenic. Thus, the peptides described herein are useful for preventing, inhibiting and killing pathogenic bacteria while not substantially harming other bacterial flora.

As shown herein, a single peptide (HBCA2) kills pathogenic bacteria such as E. coli (EHEC), K. pneunomiae, P. aeruginosa, S. enteritidis, L. monocytogenes, S. aureus (MRSA), and E. faecalis. Additionally, it kills two other major pathogens: Gardnerella vaginalis, associated with bacterial vaginosis; and Porphyromonas gingivalis, an oral anaerobe associated with periodontitis. The peptide thus has utility as a safe and effective antimicrobial agent in various applications towards stopping and preventing infections, blocking undesirable inoculations and preventing the growth of undesirable bacteria in foods, beverages, consumer products, cosmetics and health care products. While this peptide lyses these gram-positive and gram-negative pathogenic bacteria, it has no detected activity against commensal Lactobacillus and Bifidobacterium strains commonly found in the infant and adult gut. This combination of antimicrobial efficacy against pathogens and inertness relative to beneficial commensal organisms, makes the peptide a therapeutic agent for humans and animals, including but not limited to livestock and production animals, companion animals at risk of infections, but for whom antibiotics lead to permanent disruption of their protective endogenous microbial communities in the intestine and other body sites.

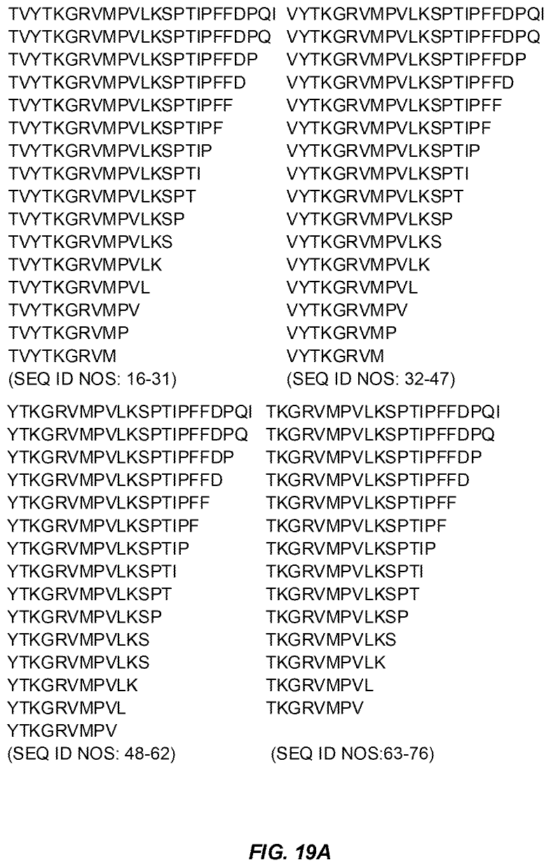

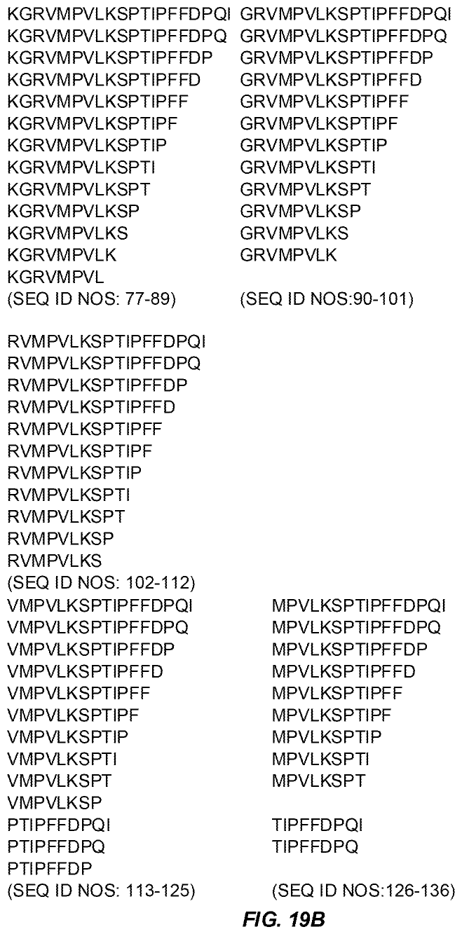

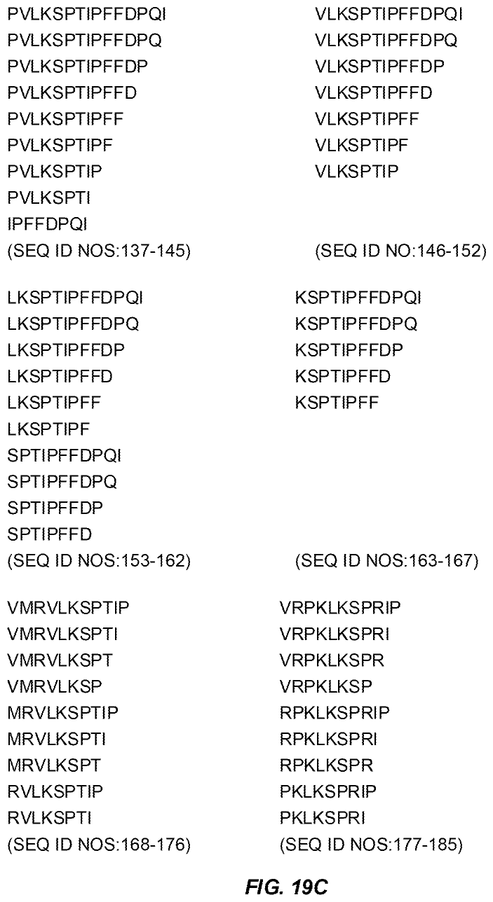

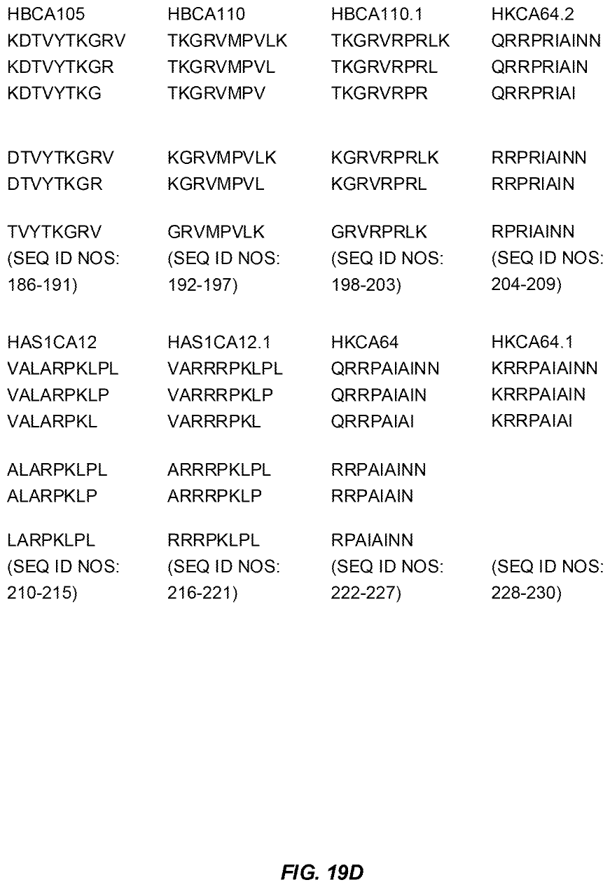

Exemplary polypeptides described herein include but are not limited to polypeptides having, or comprising a sequence, at least 70% identity, or at least 80%, 90%, 95%, 96%, 97%, 98%, 99% or 100% identity, or 0, 1, 2, 3, 4, or 5 amino acid changes compared, to RVMPVLKSPTIP (SEQ ID NO:1) RVMRVLKSPTIP (SEQ ID NO:3) or RVRPKLKSPRIP (SEQ ID NO:4). In some embodiments, the polypeptide comprises RV(M/R)(P/R)(V/K)LKSP(T/R)IP (SEQ ID NO: 231) or an active fragment thereof Alternatively, active fragments of SEQ ID NO:1, SEQ ID NO:3 or SEQ ID NO:4 can be used, including for example, fragments that are amino or carboxyl-terminus truncations lacking, e.g., 1, 2, 3, 4, 5, or more amino acids compared to SEQ ID NO:1, SEQ ID NO:3 or SEQ ID NO:4. In some embodiments, the active fragment comprises at least 8, 9, 10, or 11 amino acids of SEQ ID NO:1, SEQ ID NO:3 or SEQ ID NO:4. In some embodiments, the polypeptide comprises a fragment of SEQ ID NO:1, SEQ ID NO:3 or SEQ ID NO:4 selected from a sequence listed in FIG. 19A-C. In some embodiments, the anti-bacterial peptide can comprise SEQ ID NO:2.

In some embodiments, the anti-bacterial peptides comprise a fragment of, but not the full-length, of human β-casein (e.g., UniProtKB-P05814). Exemplary anti-bacterial peptides can comprise 8-200 amino acids of human casein, e.g., 8-150, 8-100, 8-50, 8-30, 10-20, 12-20, 12-50, 12-100, 12-150 amino acids of human casein and comprising, for example, SEQ ID NO:1, SEQ ID NO:3, SEQ ID NO:4, SEQ ID NO:5, SEQ ID NO:6 or SEQ ID NO:7.

In some embodiments, the anti-bacterial peptides comprise a fragment of, but not the full-length, of human αS1-casein (e.g., UniProtKB-P47710). Exemplary anti-bacterial peptides can comprise 8-200 amino acids of human casein, e.g., 8-150, 8-100, 8-50, 8-30, 10-20, 12-20, 12-50, 12-100, 12-150 amino acids of human casein and comprising, for example, SEQ ID NO:8 or SEQ ID NO:9.

In some embodiments, the anti-bacterial peptides comprise a fragment of, but not the full-length, of human κ-casein (e.g., UniProtKB-P07498). Exemplary anti-bacterial peptides can comprise 8-200 amino acids of human casein, e.g., 8-150, 8-100, 8-50, 8-30, 10-20, 12-20, 12-50, 12-100, 12-150 amino acids of human casein and comprising, for example, SEQ ID NO:10, SEQ ID NO:11 or SEQ ID NO:12.

The length of the anti-bacterial peptides described herein can be, any length, for example, 8-200 amino acids of human casein, e.g., 8-150, 8-100, 8-50, 8-30, 10-20, 12-20, 12-50, 12-100, or 12-150 amino acids. In some embodiments, part of the anti-bacterial peptide sequence will be an amino acid sequence that is heterologous to the human β-casein fragment. As one example, SEQ ID NO:1, SEQ ID NO:3 or SEQ ID NO:4 could be linked to one or more heterologous amino acids, for example as discussed below.

In some embodiments, the amino acid sequence is KDTVYTKGRV (SEQ ID NO:5), TKGRVMPVLK (SEQ ID NO:6), TKGRVRPRLK (SEQ ID NO:7), VALARPKLPL (SEQ ID NO:8), VARRRPKLPL (SEQ ID NO:9), QRRPAIAINN (SEQ ID NO:10), KRRPAIAINN (SEQ ID NO:11), QRRPRIAINN (SEQ ID NO:12).

The polypeptides can be generated by any method. For example, in some embodiments the protein can be purified from naturally-occurring sources, synthesized, or more typically can be made by recombinant production in a prokaryotic or eukaryotic cell engineered to produce the protein. Exemplary expression systems include various yeast, insect, and mammalian expression systems. In some embodiments, the peptide is generated by enzymatic cleavage and separation from milk or milk products or milk proteins or the release of this peptide from a combination of a polypeptide containing the peptide within its overall sequence together with enzymes or chemical catalysts that release the peptide from the protein.

The proteins as described herein can be fused to one or more fusion partners and/or heterologous amino acids to form a fusion protein. Fusion partner sequences can include, but are not limited to, amino acid tags, non-L (e.g., D-) amino acids or other amino acid mimetics to extend in vivo half-life and/or protease resistance, targeting sequences or other sequences. In some embodiments, functional variants or modified forms of the proteins include fusion proteins of an antibacterial protein as described herein and one or more fusion domains. Exemplary fusion domains include, but are not limited to, polyhistidine, Glu-Glu, glutathione S transferase (GST), thioredoxin, protein A, protein G, an immunoglobulin heavy chain constant region (Fc), maltose binding protein (MBP), and/or human serum albumin (HSA). A fusion domain or a fragment thereof may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QLAexpress™ system (Qiagen) useful with (HIS6 (SEQ ID NO: 232)) fusion partners. As another example, a fusion domain may be selected so as to facilitate detection of the proteins. Examples of such detection domains include the various fluorescent proteins (e.g., GFP) as well as “epitope tags,” which are usually short peptide sequences for which a specific antibody is available. Epitope tags for which specific monoclonal antibodies are readily available include FLAG, influenza virus haemagglutinin (HA), and c-Myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, a protein is fused with a domain that stabilizes the protein in vivo (a “stabilizer” domain). By “stabilizing” is meant anything that increases the life time of the protein in the circulating blood, regardless of whether this is because of decreased destruction, decreased clearance by the kidney, or other pharmacokinetic effect. Fusions with the Fc portion of subtypes IgG1 or IgG2a immunoglobulin are known to confer desirable pharmacokinetic properties on a wide range of proteins. See, e.g., U.S. Patent Publication No. 2014/056879. Certain mutations of these Fc portions of these IgGs confer even better pharmacokinetic properties. Generation of mutated variants of the human form of the MHC class I-related receptor, FcRn, with increased affinity for mouse immunoglobulin G, as described in Zhou J, Johnson JE, Ghetie V, Ober RJ, Ward ES. J Mol Biol. 2003 Sep 26;332(4):901-13. Likewise, fusions to human serum albumin can confer desirable properties. Other types of fusion domains that may be selected include multimerizing (e.g., dimerizing, tetramerizing) domains and functional domains (that confer an additional biological function, as desired). Fusions may be constructed such that the heterologous peptide is fused at the amino terminus of a polypeptide and/or at the carboxyl terminus of a polypeptide.

In some embodiments, the polypeptides as described herein will comprise at least one non-naturally encoded amino acid. In some embodiments, a polypeptide comprises 1, 2, 3, 4, or more unnatural amino acids. Methods of making and introducing a non-naturally-occurring amino acid into a protein are known. See, e.g., U.S. Pat. Nos. 7,083,970; and 7,524,647. The general principles for the production of orthogonal translation systems that are suitable for making proteins that comprise one or more desired unnatural amino acid are known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004; WO 2006/110182, filed Oct. 27, 2005, entitled “ORTHOGONAL TRANSLATION COMPONENTS FOR THE VIVO INCORPORATION OF UNNATURAL AMINO ACIDS” and WO 2007/103490, filed Mar. 7, 2007, entitled “SYSTEMS FOR THE EXPRESSION OF ORTHOGONAL TRANSLATION COMPONENTS IN EUBACTERIAL HOST CELLS.” For discussion of orthogonal translation systems that incorporate unnatural amino acids, and methods for their production and use, see also, Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang, et al., (2006) “Expanding the Genetic Code.” Annu Rev Biophys Biomol Struct 35: 225-249; Deiters, et al, (2005) “In vivo incorporation of an alkyne into proteins in Escherichia coli.” Bioorganic & Medicinal Chemistry Letters 15: 1521-1524; Chin, et al., (2002) “Addition of p-Azido-L-phenylalanine to the Genetic Code of Escherichia coli.” J Am Chem Soc 124: 9026-9027; and International Publication No. W02006/034332, filed on Sep. 20, 2005. Additional details are found in U.S. Pat. Nos. 7,045,337; 7,083,970; 7,238,510; 7,129,333; 7,262,040; 7,183,082; 7,199,222; and 7,217,809.

A non-naturally encoded amino acid is typically any structure having any substituent side chain other than one used in the twenty natural amino acids. Because non-naturally encoded amino acids typically differ from the natural amino acids only in the structure of the side chain, the non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

Another type of modification that can optionally be introduced into the proteins (e.g. within the polypeptide chain or at either the N- or C-terminal), e.g., to extend in vivo half-life, is PEGylation or incorporation of long-chain polyethylene glycol polymers (PEG). Introduction of PEG or long chain polymers of PEG increases the effective molecular weight of the present polypeptides, for example, to prevent rapid filtration into the urine. In some embodiments, a Lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are well known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which is incorporated herein by reference.

Another alternative approach for incorporating PEG or PEG polymers through incorporation of non-natural amino acids (as described above) can be performed with the present polypeptides. This approach utilizes an evolved tRNA/tRNA synthetase pair and is coded in the expression plasmid by the amber suppressor codon (Deiters, A, et al. (2004). Bio-org. Med. Chem. Lett. 14, 5743-5). For example, p-azidophenylalanine can be incorporated into the present polypeptides and then reacted with a PEG polymer having an acetylene moiety in the presence of a reducing agent and copper ions to facilitate an organic reaction known as “Huisgen [3+2]cycloaddition.”

In certain embodiments, specific mutations of proteins can be made to alter the glycosylation of the polypeptide. Such mutations may be selected to introduce or eliminate one or more glycosylation sites, including but not limited to, O-linked or N-linked glycosylation sites. In certain embodiments, the proteins have glycosylation sites and patterns unaltered relative to the naturally-occurring proteins. In certain embodiments, a variant of proteins includes a glycosylation variant wherein the number and/or type of glycosylation sites have been altered relative to the naturally-occurring proteins. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created.

Exemplary proteins variants include cysteine variants wherein one or more cysteine residues are deleted from or substituted for another amino acid (e.g., serine) relative to the amino acid sequence of the naturally-occurring proteins. In certain embodiments, cysteine variants may be useful when proteins must be refolded into a biologically active conformation such as after the isolation of insoluble inclusion bodies. In certain embodiments, cysteine variants have fewer cysteine residues than the native polypeptide. In certain embodiments, cysteine variants have an even number of cysteine residues to minimize interactions resulting from unpaired cysteines.

The peptides described herein can be used to inhibit or kill susceptible bacteria in any setting. In some embodiments, the peptides can be applied (e.g., as a liquid or aerosol spray) to a surface or inanimate object to sterilize the surface or object. Alternatively, the peptide can be applied to or administered to a human or non-human animal or to a plant having or at risk of having a susceptible bacteria. In some embodiments, the antibacterial peptide is added to infant formula, infant supplements or infant fortifiers. In some embodiments, the antibacterial peptide is administered to premature or full-term infants. In some embodiments, administration of the peptide prevents or reduces the occurrence necrotizing enterocolitis (NEC).

In some embodiments, the antibacterial peptide is administered to a human at risk for infection such as a human who will receive or has received an organ transplant or a human who has received chemotherapy. In some embodiments, the human is bone marrow transplant patient or otherwise has a suppressed immune response.

As noted herein, bacteria susceptible to the peptide include but are not necessarily limited to Escherichia coli (e.g., enterohemrrhagic E. coli (EHEC), including but not limited to strain O157:H7), Pseudomonas aeruginosa, Klebsiella pneumoniae, Staphylococcus aureus (MRSA) Listeria monocytogenes, Enterococcus faecalis, Gardnerella vaginalis and S. enteriditis. Notably, the peptides did not harm Lactobacillus crispatus, L. gasseri or L. jensenni strains tested. These latter species are considered part of the healthy microbiome (e.g., vaginal microbiome).

The compositions administered may further comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions (see, e.g., Remington's Pharmaceutical Sciences, 17th ed., 1989).

Formulations suitable for administration include aqueous and non-aqueous solutions, isotonic sterile solutions, which can contain antioxidants, buffers, bacteriostats, antifungal agents and solutes that render the formulation isotonic, and aqueous and non-aqueous sterile suspensions that can include suspending agents, solubilizers, thickening agents, stabilizers, and preservatives. The formulations of compounds can be presented in unit-dose or multi-dose sealed containers, such as ampoules and vials. Solutions and suspensions can be prepared from sterile powders, granules, and tablets.

The formulations can comprise one of more anti-bacterial peptide as described herein and at least one or more of one or more acidic buffer, e.g., to maintain the pH within a range of 4-6, e.g., 5-5.5. Exemplary acidic buffers can include but are not limited to of L-lactic acid, D-lactic acid, citric acid, malic acid, tartaric acid, phosphoric acid, acetic acid, propionic acid, acetic acid, butyric acid, indole lactic acid, indole acetic acid, indole propionic acid, indole acrylic acid, indole aldehyde, or indole ethanol.

Administration to a human or non-human animal can be for example intravaginal, oral, rectal, intravenous, via inhalation, nasal, rectal, intraperitoneal, parenteral, intramuscular, subcutaneous, topical or transdermal. In some embodiments, the antibacterial peptide is administered as part of a mouthwash, ointment, or other composition. In some embodiments, the antibacterial peptide is used as, or as part of, an adherent coating for skin, oral and gastric devices including but not limited to casts, bandages, supporting prosthetics, temporary dentures, teeth guards, temporary fillings, crowns and cosmetic appliances, nasogastric tubes, catheters and wearable technologies including diagnostic, monitoring and imaging devices. In some embodiments, the antibacterial peptide is used as part of companion animal restraints, bandages or identification devices. In some embodiments, the antibacterial peptide is used as a preventative agent against deleterious infections, inoculations and biofilm formation in environmental surfaces and fluids including but not limited to hospital, domestic, farm and industrial structures.

In some embodiments, the antibacterial peptide is inserted into the genome of a plant and expressed in one or more plant tissues to protect either the plant itself or inhabitants on that plant or consumers of that plant.

In some embodiments, the antibacterial peptide is expressed from the genome of animals, avians, insects or reptiles. In some embodiments, the antibacterial peptide is expressed in a tissue specific manner so as to alter these tissues (eggs, flesh, biofluids) by increasing the concentration of the peptide in those tissues. In some embodiments, the tissues are used as food products for humans or non-human animals.

One particular area of interest is bacterial vaginosis, a common recurrent infection in women of reproductive age and is associated with severe consequences such as spontaneous abortions, premature births, HIV and STD transmissions and reproductive loss in acute cases. In some embodiments, the antibacterial peptide is included in a therapeutic product for the treatment of bacterial vaginosis. The antibacterial peptide's activity against G. vaginalis and selectivity where it is not harmful to healthy vaginal lactobacilli allow for its use as a topical treatment to alter the course of this disease.

The antibacterial peptides can be administered directly to the human or non-human animal. Alternatively, a polynucleotide encoding the peptide can be expressed from the genome of a microorganism including but not limited to commensal bacteria of the oral, skin, intestinal, mucosal or reproductive tract in humans and animals and thence expressed with or without genetic regulation. In some embodiments, an expression cassette comprising a promoter operably linked to the polynucleotide encoding the peptide is introduced into a microorganism (e.g., a bacterial strain) that will in turn express the peptide before or after (or both) the microorganism is administered to the human or non-human animal. In some embodiments, the promoter is heterologous to the polynucleotide encoding the peptide. In some embodiments, the promoter can be inducible for example, or constitutive.

### EXAMPLE

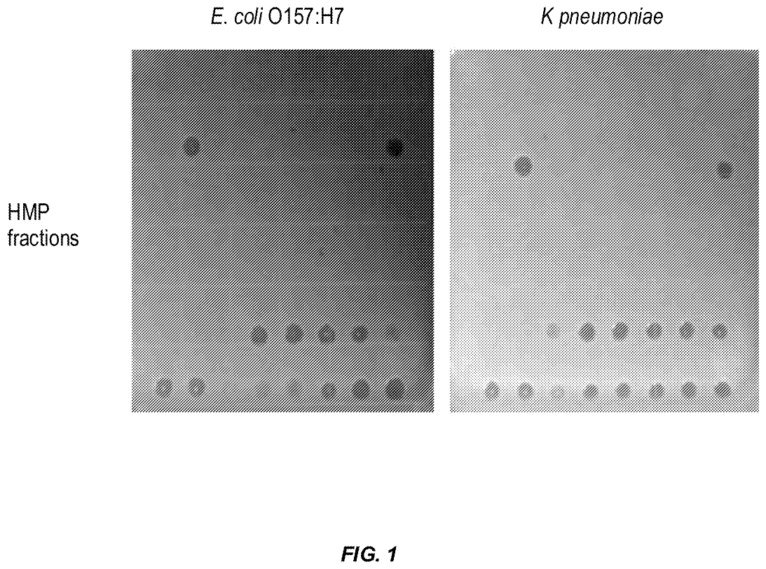

Human milk subjected to flash fractionation chromatography resulted in various peptides fractions. The fractions were dried and reconstituted in water and spotted onto Tryptic soy agar plates that were spread with 100 μL of select bacterial grown overnight in tryptic soy broth. A clear zone indicating no growth against a bacterial lawn of gram-negative pathogens like Escherichia coli O157:H7 and Klebsiella pneumoniae demonstrated bactericidal activity of specific fractions (FIG. 1).

Next, the same fractions were tested against a wider range of gram-negative and gram-positive pathogens using the spot clearance method. This indicated activity against many clinically relevant pathogens, including but not limited to Priority A pathogens, as designated by the WHO. Specifically, the fractions were bactericidal against Pseudomonas aeruginosa, Salmonella enteriditis, Staphylococcus aureus MSSA, Staphylococcus aureus, MRSA, Listeria monocytogenes and Enterococcus faecalis.

### Tests of Individual Synthetic Peptides from β-casein

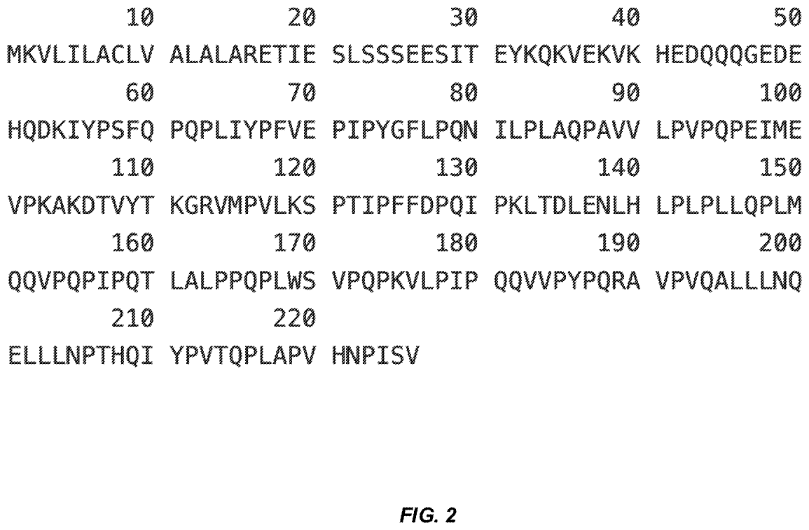

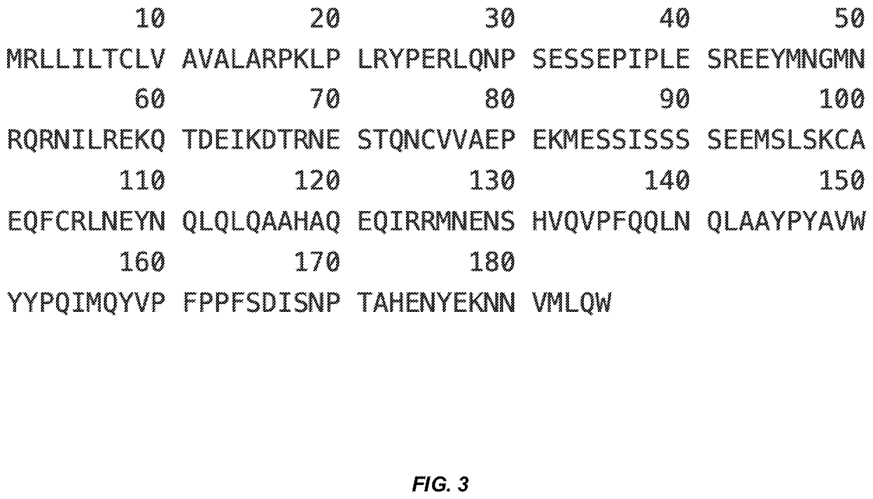

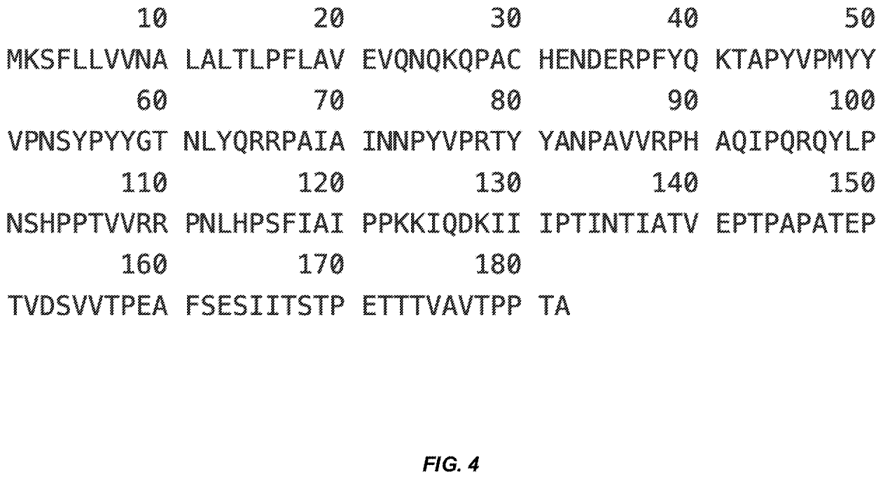

One of the most abundant proteins present in mammalian milk is β-casein, so we investigated the antimicrobial function of individual synthetic peptides derived from β-casein sequence (FIG. 2), Additionally, we tested synthetic peptides derived from αS1-casein sequence (FIG. 3) and κ-casein sequence (FIG. 4). Peptides were purchased from Genscript, Inc.

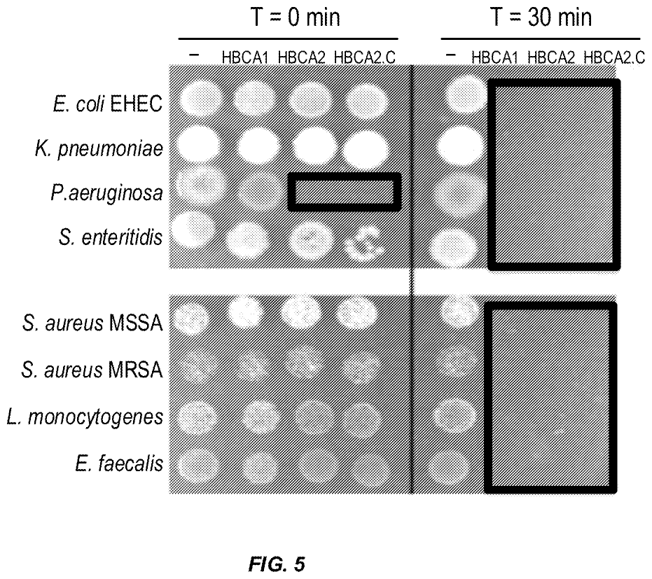

The following peptides showed antibacterial activity against the above-mentioned bacterial strains when tested via spot assays on bacterial lawns as well as direct kill assays (FIG. 5). We have named them HBCA1 and HBCA2.

I) HBCA1 (Human β-casein 1): residues 107-130

(SEQ ID NO: 2)
TVYTKGRVMPVLKSPTIPFFDPQI

Properties: 24-mer, Iso-electric point pH 10.19, Net charge at pH 7 is 2, MW: 2735.25 g/mol.
II) HBCA2 ((Human β-casein 2): residues 113-124

(SEQ ID NO: 1)
RVMPVLKSPTIP

Properties: 12-mer, Iso-electric point pH 11.39, Net charge at pH 7 is 2, MW: 1337.68 g/mol

Additionally, we tested a modified HBCA2 peptide (with C-terminal amidation) for antibacterial activity.

III) HBCA2.C (C-terminally amidated HBCA2):
Properties: 12-mer, Iso-electric point pH 14, Net charge at pH 7 is 3, MW: 1336.69 g/mol

All three peptides were tested using direct kills assays. Equal inoculums of indicated bacterial pathogens were incubated either with water (−) or 50 mg/ml of HBCA1, HBCA2 or HBCA2.C. Aliquots were spotted at T=0 min (start of incubation) and at 30 minutes post incubation (T=30 min) on LB-agar plates, and incubated at 37° C. overnight to see growth. The viability of P. aeruginosa is rapidly lost (while mixing at T0) for HBCA2 and HBCA2.C, and within 30 minutes for all other strains for the three peptides tested, as indicated by the absence of growth after overnight incubation. HBCA2.C is active similar to HBCA2 when tested in direct kill assays (FIG. 5).

These data indicate that the internal 12-mer HBCA2 is more potent in its activity than the larger HBCA1 peptide.

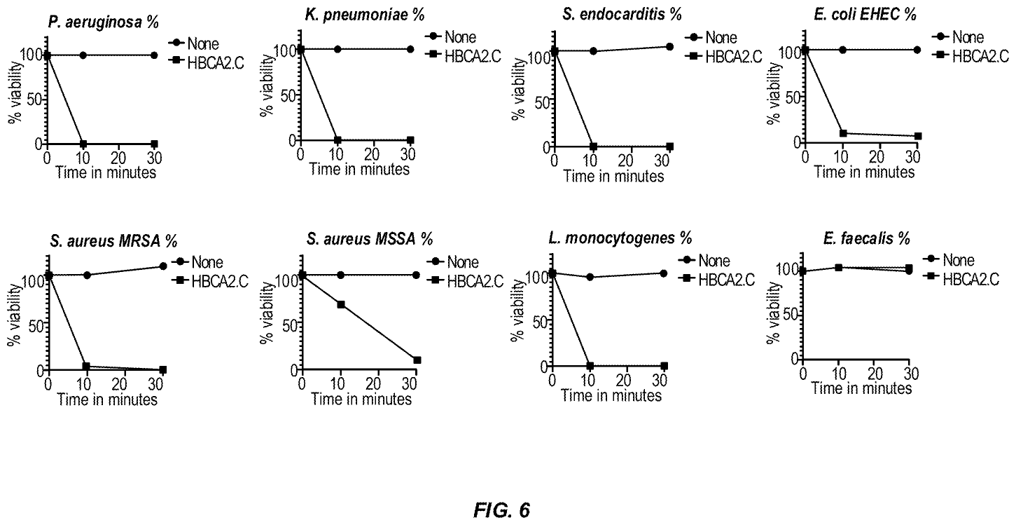

When HBCA2.C was tested at 20 mg/ml concentration in time kill assays, all of the above mentioned bacterial strains remain sensitive except for E. faecalis (FIG. 6).

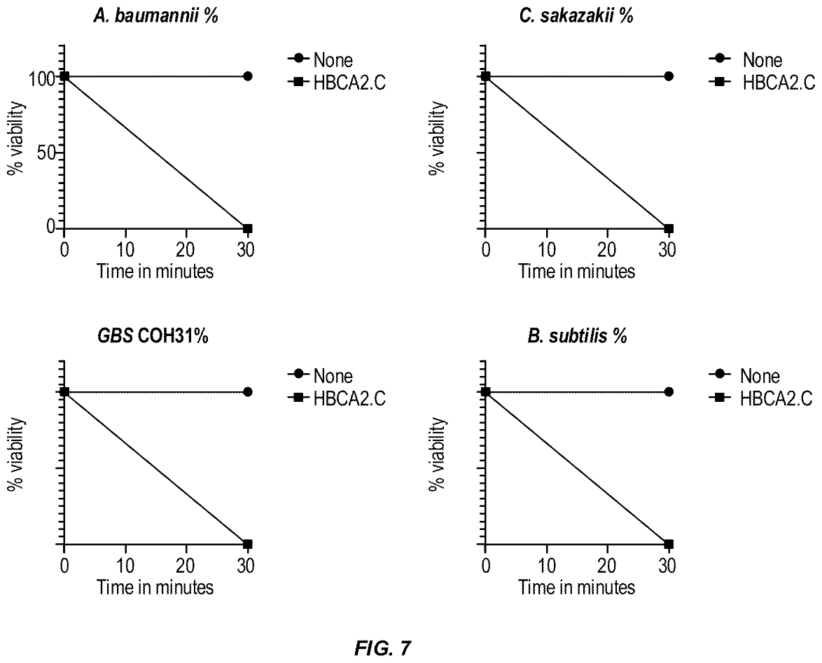

Additional bacterial strains: Acinetobacter baumannii, Chronobacter sakazakii, Group B streptococci COH31 of clinical relevance that are sensitive to HBCA2.C are shown in FIG. 7. Gram-positive spore-forming bacteria, Bacillus subtilis is also sensitive to HBCA2.C.

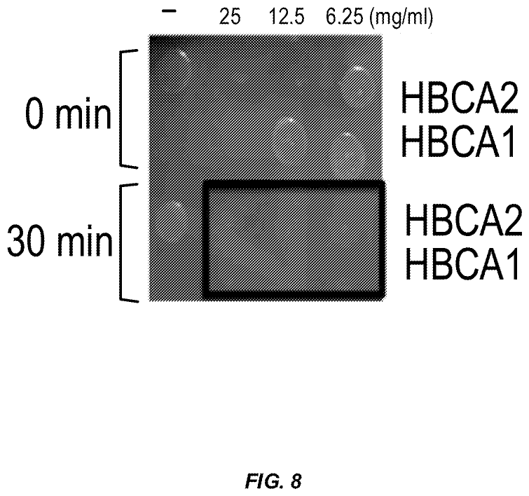

Porphyromonas gingivalis (a keystone pathogen in chronic periodontitis, and identified in the brains of Alzheimer's patients) is sensitive to HBCA1 and HBCA2 peptides. FIG. 8 shows TSA plates (5% difibrinated blood) showing loss of viability of P. gingivalis in the presence of HBCA1 and HBCA2, with HBCA2 being more potent where cells rapidly lose viability right at 0 minutes upon mixing the cells with HBCA2.

### Test of Synthetic Peptides Against Pathogens Involved in Bacterial Vaginosis and Commensal Vaginal Microbes

Next we focused on pathogens implicated in bacterial vaginosis (BV), a type of vaginal inflammation due to overgrowth of pathogens like Gardnerella vaginalis and a disruption of the healthy vaginal microbiome consisting of Lactobacillus strains, mainly, L. crispatus, L. gasseri and L. jensenni.

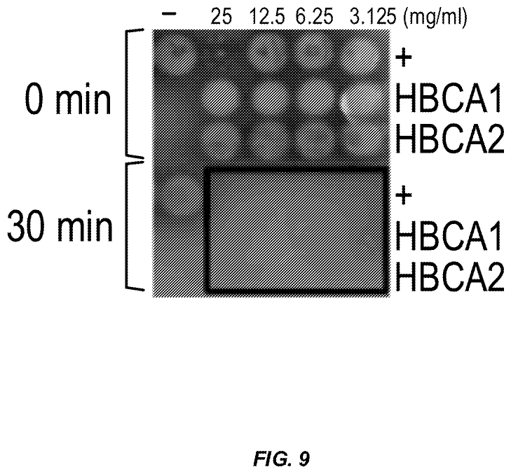

When we subjected G. vaginalis to direct kill assays using HBCA1 and HBCA2, we observed that both peptides render G. vaginalis non-viable within 30 minutes, as indicated by loss of bacterial growth on HBT bilayer Gardnerella media (FIG. 9).

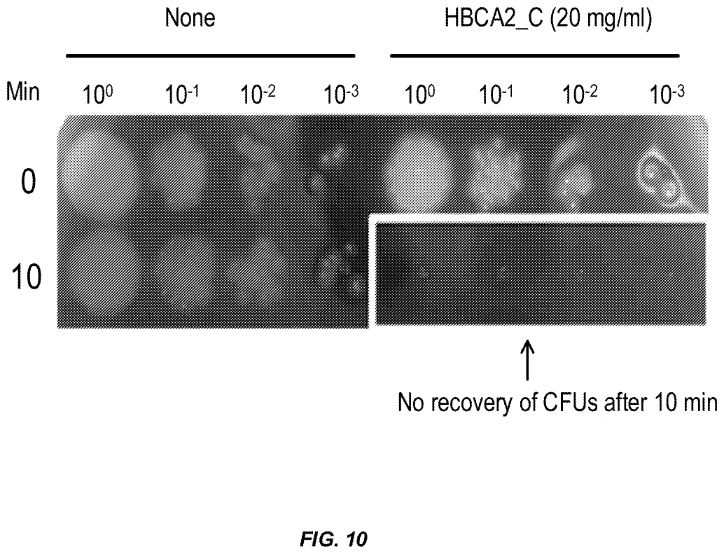

G. vaginalis is also sensitive to HBCA2.C in time kill assays at 20 mg/ml concentration (FIG. 10) as observed on HBT bilayer agar and determined by serial dilution and viable cell counts.

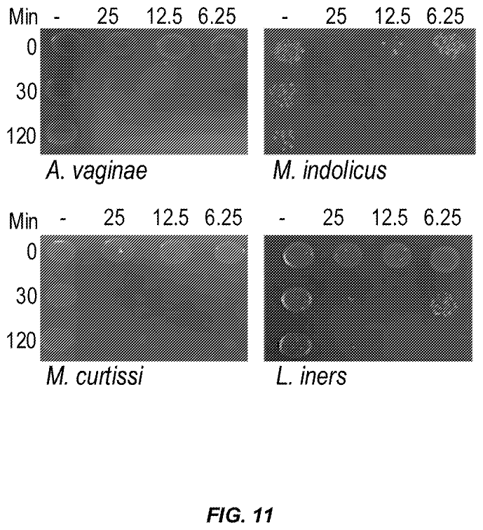

In additional confirmation of selectivity, we evaluated the direct effect of HBCA2_C on a wider range of BV associated anaerobic bacterial strains: Atopobium vaginae (Tryptic soy agar +5% sheep blood), Mageebacillus indolicus (Brucella agar +5% sheep blood) and Mobiluncus curtisii (Tryptic soy agar +5% sheep blood) and observed that these bacteria are rendered non-viable within 30 minutes. Another BV associated bacterium, Lactobacillus iners (grown on Tryptic soy agar +5% sheep blood with 5% CO2), is also sensitive to HBCA2_C (FIG. 11). M. indolicus appears to be more sensitive than either A. vaginae or M. curtissi.

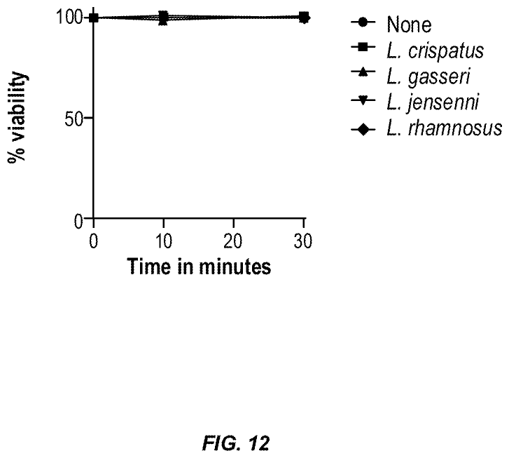

In comparison to that, all three peptides failed to show any activity towards Lactobacilli representing the healthy vaginal microbiome, in either spot assays on MRS agar plates or direct kills assays where incubation was carried on for over 180 minutes. FIG. 12 shows the time kill assays with no loss in viability of healthy vaginal lactobacilli at 20 mg/ml of HBCA2.C. Additionally, L. rhamnosus (commercial probiotic LGG) is insensitive to HBCA2.C (FIG. 12).

Thus, the activity against pathogenic bacteria and inertness against commensal bacteria can have useful applications towards the reduction of infections related to BV and have an impact on other subsequent outcomes.

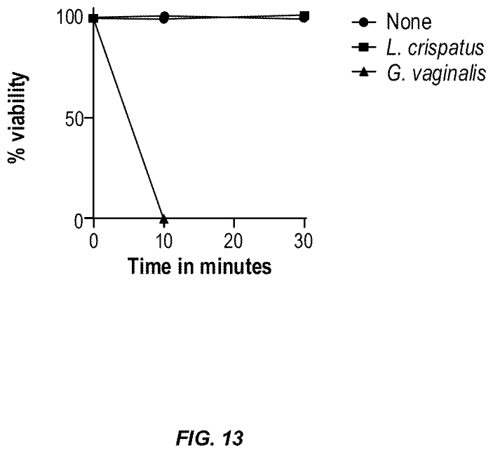

HBCA2.C selectively targets G. vaginalis in co-cultures. When starting with equivalent colony forming units of G. vaginalis and L. crispatus, HBCA2.C at 20 mg/ml eliminates the viability of G. vaginalis, but not that of L. crispatus as observed by time-kill assays (FIG. 13).

Addition of a fermentation product of Lactobacilli increases the potency of HBCA2.C

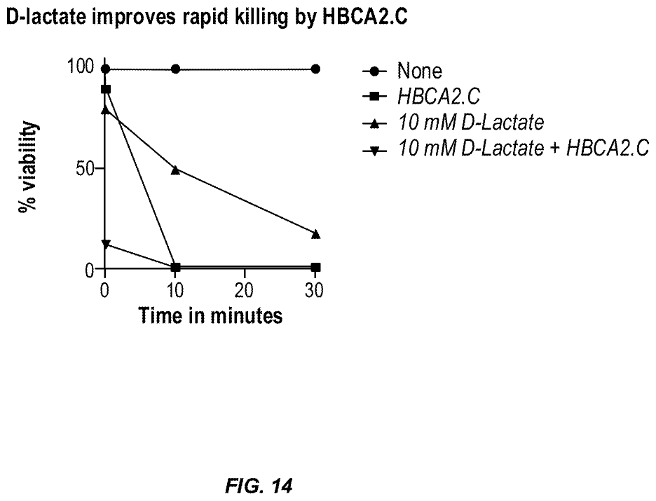

Presence of D-lactate (a short-chain fatty acid) produced by L. crispatus improves the efficiency of HBCA2.C in eliminating the viability of G. vaginalis. Thus, D-lactate could serve as an important co-factor in the formulation of HBCA2.C therapeutics for altering BV pathogenesis (FIG. 14).

Activity of peptides from human caseins and their variants. HBCA (peptides from human β-casein) , HAS1CA (peptides from human αS1-casein) and HKCA (peptides from human κ-casein) and their variants.

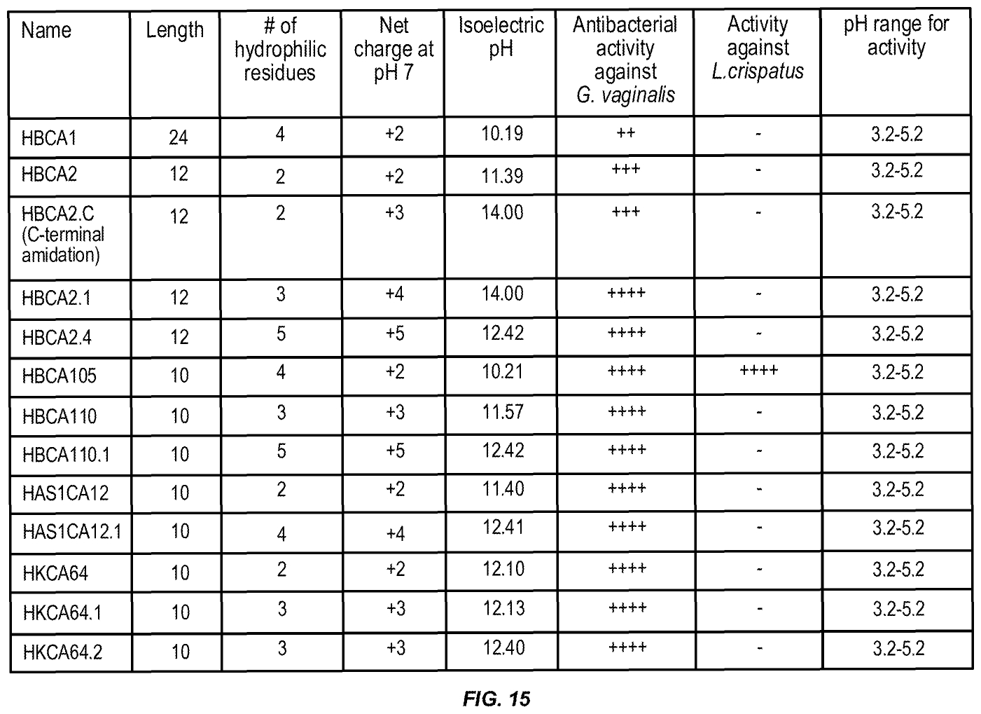

We have tested variants of HBCA2, namely HBCS2.1 and HBCA2.4, as also variants of HBCA110, namely HBCA110.1, and variants of HASICA12, namely HAS1CA12.1 and lastly of HKCA64, namely HKCA64.1 and HKCA64.2. The activity of these peptides is shown in FIG. 15. HBCA2.1 and HBCA2.4 were more potent against G. vaginalis compared to HBCA2 or HBCA2.C as shown by scoring (++++). HBCA2.1 and HBCA2.4 were more potent against G. vaginalis compared to HBCA2 or HBCA2.C as shown by the scoring (++++).

HBCA2 sequence:
(SEQ ID NO: 108)
RVMPVLKSPTIP (2 hydrophillic residues, mol wt:
1337.68)
HBCA2.1 sequence:
(SEQ ID NO: 3)
RVMRVLKSPTIP (3 hydrophillic residues, mol
wt: 1396.75)
HBCA2.4 sequence:
(SEQ ID NO: 4)
RVRPKLKSPRIP (5 hydrophillic residues, mol
wt: 1446.79)
HBCA105 sequence:
(SEQ ID NO: 186)
KDTVYTKGRV (4 hydrophillic residues, mol
wt: 1166.33)
HBCA110 sequence:
(SEQ ID NO: 74)
TKGRVMPVLK (3 hydrophillic residues, mol
wt: 1128.43)
HBCA110.1 sequence:
(SEQ ID NO: 198)
TKGRVRPRLK (5 hydrophillic residues, mol
wt: 1210.48)
HAS1CA12 sequence:
(SEQ ID NO: 210)
VALARPKLPL (2 hydrophillic residues, mol
wt: 1077.36)
HAS1CA12.1 sequence:
(SEQ ID NO: 216)
VARRRPKLPL (4 hydrophillic residues,
mol wt: 1205.50)
HKCA64 sequence:
(SEQ ID NO: 222)
QRRPAIAINN (2 hydrophillic residues, mol
wt: 1152.31)
HKCA64.1 sequence:
(SEQ ID NO: 228)
KRRPAIAINN (3 hydrophillic residues, mol
wt: 1152.35)
HKCA64.2 sequence:
(SEQ ID NO: 204)
QRRPRIAINN (3 hydrophillic residues, mol
wt: 1237.42)

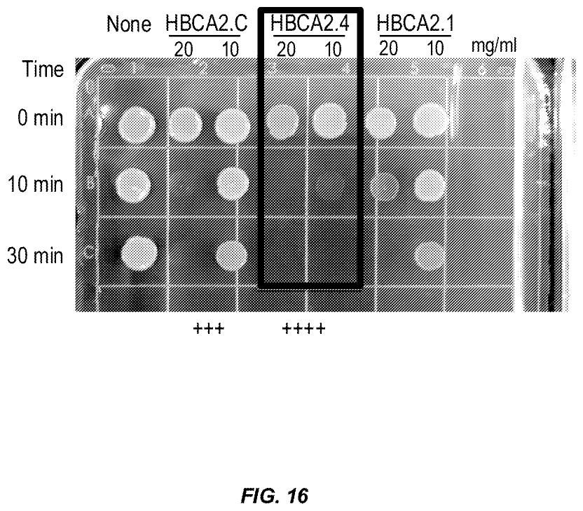

When tested against K. pneumoniae, HBCA2.4 appeared to be most potent of the HBCA peptides (FIG. 16).

Secondary structure of HBCA2.C (Circular Dichroism Spectroscopy)

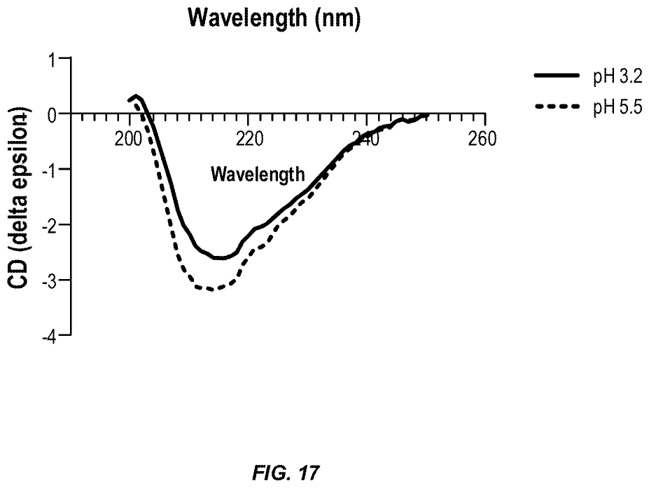

CD spectra for HBCA2.C was recorded at pH 3.2 and 5.5 (FIG. 17).

HBCA2.C was resuspended in Citric Acid-sodium citrate buffers at pH 3.2 and pH 5.5. Fitted CD spectra plots indicate random coil structure of HBCA2.C and transition towards a more helical structure with increasing pH.

Estimated secondary structure (helical) content at pH 3.2=6.7%

Estimated secondary structure (helical) content at pH 5.5=20.1%

These observations are suggestive of HBCA2.C transitioning from a random coil to helical structure upon membrane binding, and appears to be the mechanism by which HBCA2.C is activated for antimicrobial function at lower pH. Random coil structure ensured lower cytotoxicity to mammalian cells as the peptide assumes its active conformation upon bacterial binding.

Transmission Electron Microscopy (K. pneumoniae and L. rhamnosus GG).

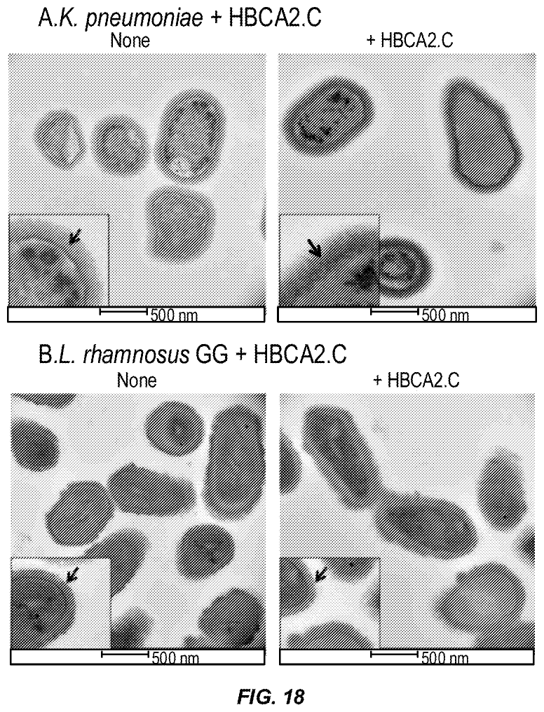

HBCA2.C eliminates the viability of K. pneumoniae, but not L. rhamnosus GG or other lactobacilli. Transition electron microscopy indicates membrane activity of HBCA2.C for K. pneumoniae (FIG. 18A), but no alterations for L. rhamnosus (FIG. 18B), confirming selective antimicrobial function.

The above disclosure is provided to illustrate the invention but not to limit its scope. Variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

1. An isolated polypeptide of 8-200 amino acids in length that comprises an amino acid sequence that has 0, 1, 2 or 3 amino changes relative to RVMPVLKSPTIP (SEQ ID NO:1), RVMRVLKSPTIP (SEQ ID NO:3), RVRPKLKSPRIP (SEQ ID NO:4), KDTVYTKGRV (SEQ ID NO:5), TKGRVMPVLK (SEQ ID NO:6), TKGRVRPRLK (SEQ ID NO:7), VALARPKLPL (SEQ ID NO:8), VARRRPKLPL (SEQ ID NO:9), QRRPAIAINN (SEQ ID NO:10), KRRPAIAINN (SEQ ID NO:11) or QRRPRIAINN (SEQ ID NO:12) or a fragment of SEQ ID NO: 1, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12

2. The isolated polypeptide of claim 1, wherein the amino acid sequence is RV(M/R)(P/R)(V/K)LKSP(T/R)IP (SEQ ID NO: 231).

3. The isolated polypeptide of claim 1, wherein the polypeptide is fused to a heterologous peptide sequence.

4. The isolated polypeptide of claim 1, wherein the amino acid sequence is SEQ ID NO:1 SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12

5. The isolated polypeptide of claim 1, wherein the polypeptide comprises TVYTKGRVMPVLKSPTIPFFDPQI (SEQ ID NO:2).

6. The isolated polypeptide of claim 1, wherein the polypeptide is 8-50 amino acids in length.

7. The isolated polypeptide of claim 1, comprising one or more D-amino acids.

8. The isolated polypeptide of claim 1, comprising a non-natural modification.

9. The isolated polypeptide of claim 8, wherein the modification is an amidation or glycosylation or PEGylation.

10. The isolated polypeptide of claim 8, wherein the modification occurs at the N- or the C-terminus of the polypeptide.

11. A composition for administration to a human or animal, the composition comprising the polypeptide of claim 1.

12. The composition of claim 11, wherein the composition is an ointment or a gel or a mouthwash, or a rinse solution, or a nutritional beverage or an adherent coating.

13. The composition of claim 11, further comprising one or more of lactic acid, citric acid, malic acid, tartaric acid, phosphoric acid, acetic acid, propionic acid, acetic acid, butyric acid, indole lactic acid, indole acetic acid, indole propionic acid, indole acrylic acid, indole aldehyde, or indole ethanol.

14. A method of administering the polypeptide of claim 1 to a human or non-human animal in an amount sufficient to inhibit a bacterial pathogen in the human or non-human animal.

15. The method of claim 14, wherein the administering comprises delivering the polypeptide to the human or non-human animal.

16. The method of claim 14, wherein the administering comprises delivering a polynucleotide encoding the polypeptide to the human or non-human animal, and the polypeptide is expressed in the human or non-human animal from the polynucleotide.

17. The method of claim 14, wherein the administering comprises delivering a cell comprising a heterologous polynucleotide encoding the polypeptide to the human or non-human animal, and the polypeptide is expressed in the cell, thereby delivering the polypeptide to the human or non-human animal.

18. The method of claim 14, wherein the administering is intravaginal, oral, rectal, intravenous, via inhalation, nasal, rectally, intraperitoneal, parenteral, intramuscular, subcutaneous, or transdermal.

19. A polynucleotide comprising a promoter operably linked to a heterologous coding sequence, wherein the coding sequence encodes the polypeptide of claim 1.

20. A method of making the polypeptide of claim 1, the method comprising expressing the polypeptide in a cell and harvesting the polypeptide from the cell.


SEQUENCE LISTING (232 sequences)

SEQ ID NO: 1 - protein, 12 aa, Homo sapiens
RVMPVLKSPTIP

SEQ ID NO: 2 - protein, 24 aa, Homo sapiens
TVYTKGRVMPVLKSPTIPFFDPQI

SEQ ID NO: 3 - protein, 12 aa, Artificial Sequence
  Description of Artificial Sequence Synthetic peptide
RVMRVLKSPTIP

SEQ ID NO: 4 - protein, 12 aa, Artificial Sequence
  Description of Artificial Sequence Synthetic peptide
RVRPKLKSPRIP

SEQ ID NO: 5 - protein, 10 aa, Homo sapiens
KDTVYTKGRV

SEQ ID NO: 6 - protein, 10 aa, Homo sapiens
TKGRVMPVLK

SEQ ID NO: 7 - protein, 10 aa, Artificial Sequence
  Description of Artificial Sequence Synthetic peptide
TKGRVRPRLK

SEQ ID NO: 8 - protein, 10 aa, Homo sapiens
VALARPKLPL

SEQ ID NO: 9 - protein, 10 aa, Artificial Sequence
  Description of Artificial Sequence Synthetic peptide
VARRRPKLPL

SEQ ID NO: 10 - protein, 10 aa, Homo sapiens
QRRPAIAINN

SEQ ID NO: 11 - protein, 10 aa, Artificial Sequence
  Description of Artificial Sequence Synthetic peptide
KRRPAIAINN

SEQ ID NO: 12 - protein, 10 aa, Artificial Sequence
  Description of Artificial Sequence Synthetic peptide
QRRPRIAINN

SEQ ID NO: 13 - protein, 226 aa, Homo sapiens
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 14 - protein, 185 aa, Homo sapiens
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 15 - protein, 182 aa, Homo sapiens
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 16 - protein, 24 aa, Homo sapiens
TVYTKGRVMPVLKSPTIPFFDPQI

SEQ ID NO: 17 - protein, 23 aa, Homo sapiens
TVYTKGRVMPVLKSPTIPFFDPQ

SEQ ID NO: 18 - protein, 22 aa, Homo sapiens
TVYTKGRVMPVLKSPTIPFFDP

SEQ ID NO: 19 - protein, 21 aa, Homo sapiens
TVYTKGRVMPVLKSPTIPFFD

SEQ ID NO: 20 - protein, 20 aa, Homo sapiens
TVYTKGRVMPVLKSPTIPFF

SEQ ID NO: 21 - protein, 19 aa, Homo sapiens
TVYTKGRVMPVLKSPTIPF

SEQ ID NO: 22 - protein, 18 aa, Homo sapiens
TVYTKGRVMPVLKSPTIP

SEQ ID NO: 23 - protein, 17 aa, Homo sapiens
TVYTKGRVMPVLKSPTI

SEQ ID NO: 24 - protein, 16 aa, Homo sapiens
TVYTKGRVMPVLKSPT

SEQ ID NO: 25 - protein, 15 aa, Homo sapiens
TVYTKGRVMPVLKSP

SEQ ID NO: 26 - protein, 14 aa, Homo sapiens
TVYTKGRVMPVLKS

SEQ ID NO: 27 - protein, 13 aa, Homo sapiens
TVYTKGRVMPVLK

SEQ ID NO: 28 - protein, 12 aa, Homo sapiens
TVYTKGRVMPVL

SEQ ID NO: 29 - protein, 11 aa, Homo sapiens
TVYTKGRVMPV

SEQ ID NO: 30 - protein, 10 aa, Homo sapiens
TVYTKGRVMP

SEQ ID NO: 31 - protein, 9 aa, Homo sapiens
TVYTKGRVM

SEQ ID NO: 32 - protein, 23 aa, Homo sapiens
VYTKGRVMPVLKSPTIPFFDPQI

SEQ ID NO: 33 - protein, 22 aa, Homo sapiens
VYTKGRVMPVLKSPTIPFFDPQ

SEQ ID NO: 34 - protein, 21 aa, Homo sapiens
VYTKGRVMPVLKSPTIPFFDP

SEQ ID NO: 35 - protein, 20 aa, Homo sapiens
VYTKGRVMPVLKSPTIPFFD

SEQ ID NO: 36 - protein, 19 aa, Homo sapiens
VYTKGRVMPVLKSPTIPFF

SEQ ID NO: 37 - protein, 18 aa, Homo sapiens
VYTKGRVMPVLKSPTIPF

SEQ ID NO: 38 - protein, 17 aa, Homo sapiens
VYTKGRVMPVLKSPTIP

SEQ ID NO: 39 - protein, 16 aa, Homo sapiens
VYTKGRVMPVLKSPTI

SEQ ID NO: 40 - protein, 15 aa, Homo sapiens
VYTKGRVMPVLKSPT

SEQ ID NO: 41 - protein, 14 aa, Homo sapiens
VYTKGRVMPVLKSP

SEQ ID NO: 42 - protein, 13 aa, Homo sapiens
VYTKGRVMPVLKS

SEQ ID NO: 43 - protein, 12 aa, Homo sapiens
VYTKGRVMPVLK

SEQ ID NO: 44 - protein, 11 aa, Homo sapiens
VYTKGRVMPVL

SEQ ID NO: 45 - protein, 10 aa, Homo sapiens
VYTKGRVMPV

SEQ ID NO: 46 - protein, 9 aa, Homo sapiens
VYTKGRVMP

SEQ ID NO: 47 - protein, 8 aa, Homo sapiens
VYTKGRVM

SEQ ID NO: 48 - protein, 22 aa, Homo sapiens
YTKGRVMPVLKSPTIPFFDPQI

SEQ ID NO: 49 - protein, 21 aa, Homo sapiens
YTKGRVMPVLKSPTIPFFDPQ

SEQ ID NO: 50 - protein, 20 aa, Homo sapiens
YTKGRVMPVLKSPTIPFFDP

SEQ ID NO: 51 - protein, 19 aa, Homo sapiens
YTKGRVMPVLKSPTIPFFD

SEQ ID NO: 52 - protein, 18 aa, Homo sapiens
YTKGRVMPVLKSPTIPFF

SEQ ID NO: 53 - protein, 17 aa, Homo sapiens
YTKGRVMPVLKSPTIPF

SEQ ID NO: 54 - protein, 16 aa, Homo sapiens
YTKGRVMPVLKSPTIP

SEQ ID NO: 55 - protein, 15 aa, Homo sapiens
YTKGRVMPVLKSPTI

SEQ ID NO: 56 - protein, 14 aa, Homo sapiens
YTKGRVMPVLKSPT

SEQ ID NO: 57 - protein, 13 aa, Homo sapiens
YTKGRVMPVLKSP

SEQ ID NO: 58 - protein, 12 aa, Homo sapiens
YTKGRVMPVLKS

SEQ ID NO: 59 - protein, 12 aa, Homo sapiens
YTKGRVMPVLKS

SEQ ID NO: 60 - protein, 11 aa, Homo sapiens
YTKGRVMPVLK

SEQ ID NO: 61 - protein, 10 aa, Homo sapiens
YTKGRVMPVL

SEQ ID NO: 62 - protein, 9 aa, Homo sapiens
YTKGRVMPV

SEQ ID NO: 63 - protein, 21 aa, Homo sapiens
TKGRVMPVLKSPTIPFFDPQI

SEQ ID NO: 64 - protein, 20 aa, Homo sapiens
TKGRVMPVLKSPTIPFFDPQ

SEQ ID NO: 65 - protein, 19 aa, Homo sapiens
TKGRVMPVLKSPTIPFFDP

SEQ ID NO: 66 - protein, 18 aa, Homo sapiens
TKGRVMPVLKSPTIPFFD

SEQ ID NO: 67 - protein, 17 aa, Homo sapiens
TKGRVMPVLKSPTIPFF

SEQ ID NO: 68 - protein, 16 aa, Homo sapiens
TKGRVMPVLKSPTIPF

SEQ ID NO: 69 - protein, 15 aa, Homo sapiens
TKGRVMPVLKSPTIP

SEQ ID NO: 70 - protein, 14 aa, Homo sapiens
TKGRVMPVLKSPTI

SEQ ID NO: 71 - protein, 13 aa, Homo sapiens
TKGRVMPVLKSPT

SEQ ID NO: 72 - protein, 12 aa, Homo sapiens
TKGRVMPVLKSP

SEQ ID NO: 73 - protein, 11 aa, Homo sapiens
TKGRVMPVLKS

SEQ ID NO: 74 - protein, 10 aa, Homo sapiens
TKGRVMPVLK

SEQ ID NO: 75 - protein, 9 aa, Homo sapiens
TKGRVMPVL

SEQ ID NO: 76 - protein, 8 aa, Homo sapiens
TKGRVMPV

SEQ ID NO: 77 - protein, 20 aa, Homo sapiens
KGRVMPVLKSPTIPFFDPQI

SEQ ID NO: 78 - protein, 19 aa, Homo sapiens
KGRVMPVLKSPTIPFFDPQ

SEQ ID NO: 79 - protein, 18 aa, Homo sapiens
KGRVMPVLKSPTIPFFDP

SEQ ID NO: 80 - protein, 17 aa, Homo sapiens
KGRVMPVLKSPTIPFFD

SEQ ID NO: 81 - protein, 16 aa, Homo sapiens
KGRVMPVLKSPTIPFF

SEQ ID NO: 82 - protein, 15 aa, Homo sapiens
KGRVMPVLKSPTIPF

SEQ ID NO: 83 - protein, 14 aa, Homo sapiens
KGRVMPVLKSPTIP

SEQ ID NO: 84 - protein, 13 aa, Homo sapiens
KGRVMPVLKSPTI

SEQ ID NO: 85 - protein, 12 aa, Homo sapiens
KGRVMPVLKSPT

SEQ ID NO: 86 - protein, 11 aa, Homo sapiens
KGRVMPVLKSP

SEQ ID NO: 87 - protein, 10 aa, Homo sapiens
KGRVMPVLKS

SEQ ID NO: 88 - protein, 9 aa, Homo sapiens
KGRVMPVLK

SEQ ID NO: 89 - protein, 8 aa, Homo sapiens
KGRVMPVL

SEQ ID NO: 90 - protein, 19 aa, Homo sapiens
GRVMPVLKSPTIPFFDPQI

SEQ ID NO: 91 - protein, 18 aa, Homo sapiens
GRVMPVLKSPTIPFFDPQ

SEQ ID NO: 92 - protein, 17 aa, Homo sapiens
GRVMPVLKSPTIPFFDP

SEQ ID NO: 93 - protein, 16 aa, Homo sapiens
GRVMPVLKSPTIPFFD

SEQ ID NO: 94 - protein, 15 aa, Homo sapiens
GRVMPVLKSPTIPFF

SEQ ID NO: 95 - protein, 14 aa, Homo sapiens
GRVMPVLKSPTIPF

SEQ ID NO: 96 - protein, 13 aa, Homo sapiens
GRVMPVLKSPTIP

SEQ ID NO: 97 - protein, 12 aa, Homo sapiens
GRVMPVLKSPTI

SEQ ID NO: 98 - protein, 11 aa, Homo sapiens
GRVMPVLKSPT

SEQ ID NO: 99 - protein, 10 aa, Homo sapiens
GRVMPVLKSP

SEQ ID NO: 100 - protein, 9 aa, Homo sapiens
GRVMPVLKS

SEQ ID NO: 101 - protein, 8 aa, Homo sapiens
GRVMPVLK

SEQ ID NO: 102 - protein, 18 aa, Homo sapiens
RVMPVLKSPTIPFFDPQI

SEQ ID NO: 103 - protein, 17 aa, Homo sapiens
RVMPVLKSPTIPFFDPQ

SEQ ID NO: 104 - protein, 16 aa, Homo sapiens
RVMPVLKSPTIPFFDP

SEQ ID NO: 105 - protein, 15 aa, Homo sapiens
RVMPVLKSPTIPFFD

SEQ ID NO: 106 - protein, 14 aa, Homo sapiens
RVMPVLKSPTIPFF

SEQ ID NO: 107 - protein, 13 aa, Homo sapiens
RVMPVLKSPTIPF

SEQ ID NO: 108 - protein, 12 aa, Homo sapiens
RVMPVLKSPTIP

SEQ ID NO: 109 - protein, 11 aa, Homo sapiens
RVMPVLKSPTI

SEQ ID NO: 110 - protein, 10 aa, Homo sapiens
RVMPVLKSPT

SEQ ID NO: 111 - protein, 9 aa, Homo sapiens
RVMPVLKSP

SEQ ID NO: 112 - protein, 8 aa, Homo sapiens
RVMPVLKS

SEQ ID NO: 113 - protein, 17 aa, Homo sapiens
VMPVLKSPTIPFFDPQI

SEQ ID NO: 114 - protein, 16 aa, Homo sapiens
VMPVLKSPTIPFFDPQ

SEQ ID NO: 115 - protein, 15 aa, Homo sapiens
VMPVLKSPTIPFFDP

SEQ ID NO: 116 - protein, 14 aa, Homo sapiens
VMPVLKSPTIPFFD

SEQ ID NO: 117 - protein, 13 aa, Homo sapiens
VMPVLKSPTIPFF

SEQ ID NO: 118 - protein, 12 aa, Homo sapiens
VMPVLKSPTIPF

SEQ ID NO: 119 - protein, 11 aa, Homo sapiens
VMPVLKSPTIP

SEQ ID NO: 120 - protein, 10 aa, Homo sapiens
VMPVLKSPTI

SEQ ID NO: 121 - protein, 9 aa, Homo sapiens
VMPVLKSPT

SEQ ID NO: 122 - protein, 8 aa, Homo sapiens
VMPVLKSP

SEQ ID NO: 123 - protein, 10 aa, Homo sapiens
PTIPFFDPQI

SEQ ID NO: 124 - protein, 9 aa, Homo sapiens
PTIPFFDPQ

SEQ ID NO: 125 - protein, 8 aa, Homo sapiens
PTIPFFDP

SEQ ID NO: 126 - protein, 16 aa, Homo sapiens
MPVLKSPTIPFFDPQI

SEQ ID NO: 127 - protein, 15 aa, Homo sapiens
MPVLKSPTIPFFDPQ

SEQ ID NO: 128 - protein, 14 aa, Homo sapiens
MPVLKSPTIPFFDP

SEQ ID NO: 129 - protein, 13 aa, Homo sapiens
MPVLKSPTIPFFD

SEQ ID NO: 130 - protein, 12 aa, Homo sapiens
MPVLKSPTIPFF

SEQ ID NO: 131 - protein, 11 aa, Homo sapiens
MPVLKSPTIPF

SEQ ID NO: 132 - protein, 10 aa, Homo sapiens
MPVLKSPTIP

SEQ ID NO: 133 - protein, 9 aa, Homo sapiens
MPVLKSPTI

SEQ ID NO: 134 - protein, 8 aa, Homo sapiens
MPVLKSPT

SEQ ID NO: 135 - protein, 9 aa, Homo sapiens
TIPFFDPQI

SEQ ID NO: 136 - protein, 8 aa, Homo sapiens
TIPFFDPQ

SEQ ID NO: 137 - protein, 15 aa, Homo sapiens
PVLKSPTIPFFDPQI

SEQ ID NO: 138 - protein, 14 aa, Homo sapiens
PVLKSPTIPFFDPQ

SEQ ID NO: 139 - protein, 13 aa, Homo sapiens
PVLKSPTIPFFDP

SEQ ID NO: 140 - protein, 12 aa, Homo sapiens
PVLKSPTIPFFD

SEQ ID NO: 141 - protein, 11 aa, Homo sapiens
PVLKSPTIPFF

SEQ ID NO: 142 - protein, 10 aa, Homo sapiens
PVLKSPTIPF

SEQ ID NO: 143 - protein, 9 aa, Homo sapiens
PVLKSPTIP

SEQ ID NO: 144 - protein, 8 aa, Homo sapiens
PVLKSPTI

SEQ ID NO: 145 - protein, 8 aa, Homo sapiens
IPFFDPQI

SEQ ID NO: 146 - protein, 14 aa, Homo sapiens
VLKSPTIPFFDPQI

SEQ ID NO: 147 - protein, 13 aa, Homo sapiens
VLKSPTIPFFDPQ

SEQ ID NO: 148 - protein, 12 aa, Homo sapiens
VLKSPTIPFFDP

SEQ ID NO: 149 - protein, 11 aa, Homo sapiens
VLKSPTIPFFD

SEQ ID NO: 150 - protein, 10 aa, Homo sapiens
VLKSPTIPFF

SEQ ID NO: 151 - protein, 9 aa, Homo sapiens
VLKSPTIPF

SEQ ID NO: 152 - protein, 8 aa, Homo sapiens
VLKSPTIP

SEQ ID NO: 153 - protein, 13 aa, Homo sapiens
LKSPTIPFFDPQI

SEQ ID NO: 154 - protein, 12 aa, Homo sapiens
LKSPTIPFFDPQ

SEQ ID NO: 155 - protein, 11 aa, Homo sapiens
LKSPTIPFFDP

SEQ ID NO: 156 - protein, 10 aa, Homo sapiens
LKSPTIPFFD

SEQ ID NO: 157 - protein, 9 aa, Homo sapiens
LKSPTIPFF

SEQ ID NO: 158 - protein, 8 aa, Homo sapiens
LKSPTIPF

SEQ ID NO: 159 - protein, 11 aa, Homo sapiens
SPTIPFFDPQI

SEQ ID NO: 160 - protein, 10 aa, Homo sapiens
SPTIPFFDPQ

SEQ ID NO: 161 - protein, 9 aa, Homo sapiens
SPTIPFFDP

SEQ ID NO: 162 - protein, 8 aa, Homo sapiens
SPTIPFFD

SEQ ID NO: 163 - protein, 12 aa, Homo sapiens
KSPTIPFFDPQI

SEQ ID NO: 164 - protein, 11 aa, Homo sapiens
KSPTIPFFDPQ

SEQ ID NO: 165 - protein, 10 aa, Homo sapiens
KSPTIPFFDP

SEQ ID NO: 166 - protein, 9 aa, Homo sapiens
KSPTIPFFD

SEQ ID NO: 167 - protein, 8 aa, Homo sapiens
KSPTIPFF

SEQ ID NO: 168 - protein, 11 aa, Artificial Sequence
  Description of Artificial Sequence Synthetic peptide
VMRVLKSPTIP

SEQ ID NO: 169 - protein, 10 aa, Artificial Sequence
  Description of Artificial Sequence Synthetic peptide
VMRVLKSPTI

SEQ ID NO: 170 - protein, 9 aa, Artificial Sequence
  Description of Artificial Sequence Synthetic peptide
VMRVLKSPT

SEQ ID NO: 171 - protein, 8 aa, Artificial Sequence
  Description of Artificial Sequence Synthetic peptide
VMRVLKSP

SEQ ID NO: 172 - protein, 10 aa, Artificial Sequence
  Description of Artificial Sequence Synthetic peptide
MRVLKSPTIP

SEQ ID NO: 173 - protein, 9 aa, Artificial Sequence
  Description of Artificial Sequence Synthetic peptide
MRVLKSPTI

SEQ ID NO: 174 - protein, 8 aa, Artificial Sequence
  Description of Artificial Sequence Synthetic peptide
MRVLKSPT

SEQ ID NO: 175 - protein, 9 aa, Artificial Sequence
  Description of Artificial Sequence Synthetic peptide
RVLKSPTIP

SEQ ID NO: 176 - protein, 8 aa, Artificial Sequence
  Description of Artificial Sequence Synthetic peptide
RVLKSPTI

SEQ ID NO: 177 - protein, 11 aa, Artificial Sequence
  Description of Artificial Sequence Synthetic peptide
VRPKLKSPRIP

SEQ ID NO: 178 - protein, 10 aa, Artificial Sequence
  Description of Artificial Sequence Synthetic peptide
VRPKLKSPRI

SEQ ID NO: 179 - protein, 9 aa, Artificial Sequence
  Description of Artificial Sequence Synthetic peptide
VRPKLKSPR

SEQ ID NO: 180 - protein, 8 aa, Artificial Sequence
  Description of Artificial Sequence Synthetic peptide
VRPKLKSP

SEQ ID NO: 181 - protein, 10 aa, Artificial Sequence
  Description of Artificial Sequence Synthetic peptide
RPKLKSPRIP

SEQ ID NO: 182 - protein, 9 aa, Artificial Sequence
  Description of Artificial Sequence Synthetic peptide
RPKLKSPRI

SEQ ID NO: 183 - protein, 8 aa, Artificial Sequence
  Description of Artificial Sequence Synthetic peptide
RPKLKSPR

SEQ ID NO: 184 - protein, 9 aa, Artificial Sequence
  Description of Artificial Sequence Synthetic peptide
PKLKSPRIP

SEQ ID NO: 185 - protein, 8 aa, Artificial Sequence
  Description of Artificial Sequence Synthetic peptide
PKLKSPRI

SEQ ID NO: 186 - protein, 10 aa, Homo sapiens
KDTVYTKGRV

SEQ ID NO: 187 - protein, 9 aa, Homo sapiens
KDTVYTKGR

SEQ ID NO: 188 - protein, 8 aa, Homo sapiens
KDTVYTKG

SEQ ID NO: 189 - protein, 9 aa, Homo sapiens
DTVYTKGRV

SEQ ID NO: 190 - protein, 8 aa, Homo sapiens
DTVYTKGR

SEQ ID NO: 191 - protein, 8 aa, Homo sapiens
TVYTKGRV

SEQ ID NO: 192 - protein, 10 aa, Homo sapiens
TKGRVMPVLK

SEQ ID NO: 193 - protein, 9 aa, Homo sapiens
TKGRVMPVL

SEQ ID NO: 194 - protein, 8 aa, Homo sapiens
TKGRVMPV

SEQ ID NO: 195 - protein, 9 aa, Homo sapiens
KGRVMPVLK

SEQ ID NO: 196 - protein, 8 aa, Homo sapiens
KGRVMPVL

SEQ ID NO: 197 - protein, 8 aa, Homo sapiens
GRVMPVLK

SEQ ID NO: 198 - protein, 10 aa, Artificial Sequence
  Description of Artificial Sequence Synthetic peptide
TKGRVRPRLK

SEQ ID NO: 199 - protein, 9 aa, Artificial Sequence
  Description of Artificial Sequence Synthetic peptide
TKGRVRPRL

SEQ ID NO: 200 - protein, 8 aa, Artificial Sequence
  Description of Artificial Sequence Synthetic peptide
TKGRVRPR

SEQ ID NO: 201 - protein, 9 aa, Artificial Sequence
  Description of Artificial Sequence Synthetic peptide
KGRVRPRLK

SEQ ID NO: 202 - protein, 8 aa, Artificial Sequence
  Description of Artificial Sequence Synthetic peptide
KGRVRPRL

SEQ ID NO: 203 - protein, 8 aa, Artificial Sequence
  Description of Artificial Sequence Synthetic peptide
GRVRPRLK

SEQ ID NO: 204 - protein, 10 aa, Artificial Sequence
  Description of Artificial Sequence Synthetic peptide
QRRPRIAINN

SEQ ID NO: 205 - protein, 9 aa, Artificial Sequence
  Description of Artificial Sequence Synthetic peptide
QRRPRIAIN

SEQ ID NO: 206 - protein, 8 aa, Artificial Sequence
  Description of Artificial Sequence Synthetic peptide
QRRPRIAI

SEQ ID NO: 207 - protein, 9 aa, Artificial Sequence
  Description of Artificial Sequence Synthetic peptide
RRPRIAINN

SEQ ID NO: 208 - protein, 8 aa, Artificial Sequence
  Description of Artificial Sequence Synthetic peptide
RRPRIAIN

SEQ ID NO: 209 - protein, 8 aa, Artificial Sequence
  Description of Artificial Sequence Synthetic peptide
RPRIAINN

SEQ ID NO: 210 - protein, 10 aa, Homo sapiens
VALARPKLPL

SEQ ID NO: 211 - protein, 9 aa, Homo sapiens
VALARPKLP

SEQ ID NO: 212 - protein, 8 aa, Homo sapiens
VALARPKL

SEQ ID NO: 213 - protein, 9 aa, Homo sapiens
ALARPKLPL

SEQ ID NO: 214 - protein, 8 aa, Homo sapiens
ALARPKLP

SEQ ID NO: 215 - protein, 8 aa, Homo sapiens
LARPKLPL

SEQ ID NO: 216 - protein, 10 aa, Artificial Sequence
  Description of Artificial Sequence Synthetic peptide
VARRRPKLPL

SEQ ID NO: 217 - protein, 9 aa, Artificial Sequence
  Description of Artificial Sequence Synthetic peptide
VARRRPKLP

SEQ ID NO: 218 - protein, 8 aa, Artificial Sequence
  Description of Artificial Sequence Synthetic peptide
VARRRPKL

SEQ ID NO: 219 - protein, 9 aa, Artificial Sequence
  Description of Artificial Sequence Synthetic peptide
ARRRPKLPL

SEQ ID NO: 220 - protein, 8 aa, Artificial Sequence
  Description of Artificial Sequence Synthetic peptide
ARRRPKLP

SEQ ID NO: 221 - protein, 8 aa, Artificial Sequence
  Description of Artificial Sequence Synthetic peptide
RRRPKLPL

SEQ ID NO: 222 - protein, 10 aa, Homo sapiens
QRRPAIAINN

SEQ ID NO: 223 - protein, 9 aa, Homo sapiens
QRRPAIAIN

SEQ ID NO: 224 - protein, 8 aa, Homo sapiens
QRRPAIAI

SEQ ID NO: 225 - protein, 9 aa, Homo sapiens
RRPAIAINN

SEQ ID NO: 226 - protein, 8 aa, Homo sapiens
RRPAIAIN

SEQ ID NO: 227 - protein, 8 aa, Homo sapiens
RPAIAINN

SEQ ID NO: 228 - protein, 10 aa, Artificial Sequence
  Description of Artificial Sequence Synthetic peptide
KRRPAIAINN

SEQ ID NO: 229 - protein, 9 aa, Artificial Sequence
  Description of Artificial Sequence Synthetic peptide
KRRPAIAIN

SEQ ID NO: 230 - protein, 8 aa, Artificial Sequence
  Description of Artificial Sequence Synthetic peptide
KRRPAIAI

SEQ ID NO: 231 - protein, 12 aa, Artificial Sequence
  Description of Artificial Sequence Synthetic peptide
  MOD_RES (3)..(3): M or R
  MOD_RES (4)..(4): P or R
  MOD_RES (5)..(5): V or K
  MOD_RES (10)..(10): T or R
RVXXXLKSPXIP

SEQ ID NO: 232 - protein, 6 aa, Artificial Sequence
  Description of Artificial Sequence Synthetic 6xHis tag
HHHHHH